(19) United States
(12) Patent Application Publication
(10) Pub. No.: US 2021/0147925 A1
(43) Pub. Date: May 20, 2021

(54) PARTITION-BASED DETERMINATION OF TARGET COPY NUMBER FOR SINGLE CELLS BY NON-ENDPOINT AMPLIFICATION

(71) Applicant: Bio-Rad Laboratories, Inc., Hercules, CA (US)

(72) Inventors: George KARLIN-NEUMANN, Palo Alto, CA (US); Douglas GREINER, Fremont, CA (US)

(73) Assignee: Bio-Rad Laboratories, Inc., Hercules, CA (US)

(21) Appl. No.: 17/098,006

(22) Filed: Nov. 13, 2020

Related U.S. Application Data
(60) Provisional application No. 62/935,538, filed on Nov. 14, 2019.

(51) Int. Cl. C12Q 1/6851; C12Q 1/6827; C12Q 1/6806; C12Q 1/6886

(52) U.S. Cl. CPC C12Q 1/6851; C12Q 1/6827; C12Q 2600/158; C12Q 1/6886; C12Q 1/6806

(57) ABSTRACT

Methods of analyzing a sample including cells and/or cell-free nuclei. In an exemplary method, partitions may be formed, with each partition including a portion of the same sample. Each partition of at least a subset of the partitions may contain only one of the cells/nuclei from the sample. Cells and/or cell-free nuclei from the sample may be lysed in the partitions. At least one amplification reaction may be performed for a target or set of targets in the partitions. Amplification data may be collected from the partitions in an exponential/linear phase of each amplification reaction. A copy number of the target or set of targets may be determined for individual partitions using the amplification data, to determine if either a duplication or deletion is present in all or a subset of the cells analyzed.

### CROSS-REFERENCE TO PRIORITY APPLICATION

This application is based upon and claims the benefit under 35 U.S.C. § 119(e) of U.S. Provisional Patent Application Ser. No. 62/935,538, filed Nov. 14, 2019, which is incorporated herein by reference in its entirety for all purposes.

### INTRODUCTION

Noninvasive test methods can fail to make a confident determination of the presence of full or partial chromosomal duplications or deletions in human samples. Chromosomal duplications can include localized (focal) gene amplifications driving cancer, or whole or partial chromosome duplications, as seen in aneuploidy of developing fetuses (e.g., common trisomies like Down syndrome). In pregnancy testing, after prenatal screening tests, high-risk individuals are tested with gold standard, invasive diagnostic methods (i.e., fluorescence in situ hybridization (FISH) and/or karyotyping). These invasive diagnostic methods require collection of fetal cells via chorionic villus sampling (CVS) or amniocentesis, each with a small risk of miscarriage (usually <1%). Newer screening methods, referred to as noninvasive prenatal testing (NIPT), assess for aneuploidy using next generation sequencing (NGS) of cell-free DNA present in maternal plasma. However, these newer methods generally require total cell-free DNA from a 10-20 mL blood sample and a sufficiently high contribution from fetal cells (the fetal fraction, or FF %) to provide an accurate result.

New noninvasive molecular screening/diagnosis methods are needed for determining the copy number of targets in single cells.

### SUMMARY

The present disclosure provides methods of analyzing a sample including cells and/or cell-free nuclei. In an exemplary method, partitions may be formed, with each partition including a portion of the sample. Each partition of at least a subset of the partitions may contain only one of the cells/nuclei from the sample. Cells and/or cell-free nuclei from the sample may be lysed in the partitions. At least one amplification reaction may be performed for a target or set of targets in the partitions. Amplification data may be collected from the partitions in an exponential/linear phase of each amplification reaction. A copy number of the target or set of targets may be determined for individual partitions using the amplification data, to determine if either a duplication or deletion is present in all or a subset of the cells analyzed.

### BRIEF DESCRIPTION OF THE DRAWINGS

FIG. 1 is a flowchart listing exemplary steps that may be performed in a partition-based amplification method of analyzing a sample including cells or nuclei, to determine a copy number of at least one target or set of targets for individual cells or nuclei of the sample.

FIG. 2 is a schematic diagram illustrating aspects of an exemplary partition-based amplification method performed for two different targets or target sets on a sample including maternal cells and fetal cells, where the maternal cells are disomic for both of the targets or target sets, where the fetal cells are trisomic for only one of the targets or target sets, and where photoluminescence is detected from partitions in two different wavelength regimes to assess target amplification.

FIG. 3 is a conceptual histogram showing exemplary fluorescence that may be detected from partitions in the method of FIG. 2, where the intensity of the fluorescence corresponds to the copy number of a target in a chromosome that is disomic in maternal cells and trisomic in fetal cells. The number of copies of the first target, and the number of cells, present in partitions of each distinct partition group of the histogram are indicated.

FIG. 4 is conceptual scatterplot showing exemplary fluorescence in two different wavelength regimes (A and B) that may be detected from partitions in the method of FIG. 2, where the intensity of fluorescence A corresponds to the copy number of a first target in a chromosome that is disomic in maternal cells and trisomic in fetal cells, and where the intensity of fluorescence B corresponds to the copy number of a second target in a chromosome that is disomic in both maternal cells and fetal cells. The number of copies of the first target, and the number of cells, present in partitions of each partition cluster are indicated.

FIG. 5 is a graph plotting the intensity of FAM fluorescence measured from seven separate sets of droplets each pre-loaded with a different amount of FAM dye (i.e., 50 nM, 100 nM, 200 nM, etc.).

FIG. 6 is a graph plotting the amplitude of FAM fluorescence, as an indicator of target amplification, detected from individual droplets (events) as the number of PCR cycles increases, for four separate sets of droplets.

FIG. 7 shows a pair of graphs comparing droplet-based amplification assays using a supercoiled template (on the left) or a linearized form of the template (on the right) as the source of the same target sequence, where the amplitude of FAM fluorescence is detected from a series of droplets (events) and is directly related to the amount of amplification of the target sequence.

FIG. 8 is a two-dimensional fluorescence scatterplot of amplification data collected from droplets containing wild-type (WT) and mutant (G12D) N-Ras target sequences in various combinations, where the wild-type and mutant target sequences are detected as increases in HEX and FAM fluorescence, respectively.

### DETAILED DESCRIPTION

The present disclosure provides methods of analyzing a sample including cells and/or cell-free nuclei. In an exemplary method, partitions may be formed, with each partition including a portion of the sample. Each partition of at least a subset of the partitions may contain only one of the cells/nuclei from the sample. Cells and/or cell-free nuclei from the sample may be lysed in the partitions. At least one amplification reaction may be performed for a target or set of targets in the partitions. Amplification data may be collected from the partitions in an exponential/linear phase of each amplification reaction. A copy number of the target or set of targets may be determined for individual partitions using the amplification data, to determine if either a duplication or deletion is present in all or a subset of the cells analyzed.

The methods of the present disclosure combine the accuracy benefits of single-cell determination, as in fluorescence in situ hybridization (FISH), with the simplicity of a single-cell amplification approach. Prenatal testing may be performed relatively noninvasively with fetal cells obtained from maternal blood. The copy number per cell of one or more selected nucleic acid targets (either DNA or RNA) may be measured robustly. The methods may be applied to noninvasive prenatal testing (NIPT) and/or noninvasive prenatal diagnosis (NIPD). In other words, prenatal screening or diagnosis using the methods may determine if any partial/complete chromosome deletions or duplications (e.g., Chr 21 in Down Syndrome) are present in the cells isolated from maternal blood. These methods may be much less sensitive to the percentage of fetal cells in the maternal blood (i.e., the fetal fraction) since each cell is scored individually for a trisomy. The methods also may be applied to oncology testing/diagnosis, where isolated circulating tumor cells (CTCs) may be assessed to determine whether a gene amplification is present in a tumor (e.g., HER2 amplification in metastatic breast cancer or FGFR2 amplification in a gastrointestinal stromal tumor (GIST)). A mixed cell sample may be analyzed (e.g., fetal cells among maternal lymphocytes, or CTCs among lymphocytes). Cells having an abnormal copy number (CN) of a target or set of targets may be identified as a distinct group of partitions separated from partitions that received normal cells, in one-dimensional or two-dimensional partition plots.

Further aspects of the present disclosure are described in the following sections: (I) definitions, (II) method overview, (III) examples, and (IV) selected aspects.

### I. DEFINITIONS

Technical terms used in this disclosure have meanings that are commonly recognized by those skilled in the art. However, the following terms may be further defined as follows.

Amplicon—a product of an amplification reaction. An amplicon may be generated by amplification of a target, such that the amplicon corresponds to the target (i.e., matches and/or is complementary to the target). However, the sequence of the amplicon, such as at primer binding sites, may not exactly match and/or may not be perfectly complementary to the sequence of the target.

Amplification—a process whereby multiple copies are made of an amplicon corresponding to a target. The process interchangeably may be called target amplification. Amplification may generate an exponential increase in the number of copies as amplification proceeds. Typical amplifications may produce a greater than 1,000-fold increase in the number of copies of an amplicon. Exemplary amplification reactions for the methods disclosed herein may include the polymerase chain reaction (PCR) or ligase chain reaction (LCR), each of which is driven by thermal cycling. The methods also or alternatively may use other amplification reactions, which may be performed isothermally, such as branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, and/or the like. Amplification may utilize a linear or circular template.

Amplification reagents—any reagents that promote target amplification. The reagents may include any combination of at least one primer pair for amplification of at least one target, at least one label for detecting amplification of the at least one target (e.g., at least one probe including a label and/or a DNA intercalating dye as a label), at least one polymerase enzyme and/or ligase enzyme (which may be heat-stable), and nucleoside triphosphates (dNTPs and/or NTPs), among others.

Droplet—a small volume of liquid encapsulated by an immiscible fluid (e.g., encapsulated by an immiscible liquid, which may form a continuous phase of an emulsion). The immiscible liquid may include oil and/or may be composed predominantly of oil. Droplets for the methods disclosed herein may, for example, have an average size of less than about 500 nL, 100 nL, 10 nL, or 1 nL, among others.

Label—an identifying and/or distinguishing marker or identifier associated with a structure, such as a primer, probe, amplicon, droplet, or the like. The label may be associated covalently with the structure, such as a label that is covalently attached to an oligonucleotide, or associated non-covalently (e.g., by intercalation, hydrogen bonding, electrostatic interaction, encapsulation, etc.). Exemplary labels include optical labels, radioactive labels, magnetic labels, electrical labels, epitopes, enzymes, antibodies, etc. Optical labels are detectable optically via their interaction with light. Exemplary optical labels that may be suitable include photoluminophores, quenchers, and intercalating dyes, among others.

Light—optical radiation including ultraviolet light, visible light, and/or infrared light.

Lysis—any procedure that compromises the integrity of a cell or nucleus, and particularly the outer membrane thereof. Exemplary procedures that may be performed on a cell or nucleus to promote lysis may include heating, sonication, contact with a surfactant, catalysis of a reaction using an enzyme, and/or applying pressure through osmosis, among others. Lysis of either a whole cell (containing a nucleus) or a cell-free nucleus may release genomic DNA (and RNA/protein) from the nucleus (and/or cytoplasm) and may disrupt chromatin structure formed with the genomic DNA, optionally separating histones from the genomic DNA.

Lysis reagents—any reagents that promote lysis of a cell/nucleus and/or increase accessibility of a target sequence for an amplification reaction. Lysis reagents may include a surfactant, at least one enzyme (e.g., a nuclease and/or a protease), salt, or the like.

Nucleic acid oligomer—a relatively short polynucleotide (i.e., an oligonucleotide) or a relatively short polynucleotide analogue (i.e., an oligonucleotide analogue). Exemplary analogues include peptide nucleic acids, locked nucleic acids, phosphorothiates, etc. A nucleic acid oligomer may have an unbranched (or branched) chain of conjugated units, namely, nucleotides or nucleotide analogues, each containing a base (e.g., a nucleobase). A nucleic acid oligomer may, for example, contain less than about 200, 100, 75, or 50 conjugated units, where each unit is a nucleotide or nucleotide analogue. The nucleic acid oligomer may be chemically synthesized or biosynthesized, among others. The nucleic acid oligomer may be labeled with at least one label, which may be conjugated to the chain and considered part of the oligomer. The at least one label may include at least one photoluminophore and thus may be a photoluminescent label. Each label may be conjugated to the chain of the nucleic acid oligomer at any suitable position, including a 5′-end, 3′-end, or intermediate 5′- and 3′-ends.

Partitions—a set of liquid volumes that are isolated from one another. Each liquid volume may contain a portion of the same sample-containing fluid. The liquid volumes may be separated from one another using an immiscible liquid (e.g., oil), walls of a device(s), or a combination thereof, among others. Accordingly, the liquid volumes may be droplets of an emulsion, or volumes held by wells, chambers (e.g., nanochambers having a capacity of less than 1 μL), or tubes (e.g., microtubes having a diameter of less than 1 mm), among others. The liquid volumes may be of substantially the same size and/or may contain substantially the same amount of fluid.

Photoluminescence—emission of light, where the emission is induced by electromagnetic radiation. Photoluminescence may be produced by any form of matter in response to absorption of photons of electromagnetic radiation, such as light. Exemplary forms of photoluminescence include fluorescence and phosphorescence, among others.

Photoluminophore—a species (such as a label) capable of emitting light in response to absorption of electromagnetic radiation. Accordingly, a photoluminophore may, for example, be a fluorophore or a phosphor. Suitable photoluminophores may include a dye, such as FAM, VIC, HEX, ROX, TAMRA, JOE, Cyanine-3, or Cyanine-5 dye, or the like.

Probe—a labeled nucleic acid oligomer (an oligonucleotide or analogue thereof) configured to report amplification of a target. A probe may be a photoluminescent probe including a nucleic acid oligomer labeled with a photoluminophore. A probe may be configured to hybridize with at least a portion of an amplicon generated by target amplification. The probe (e.g., a hydrolysis probe) may be configured to hybridize with at least a portion of an amplicon during an amplification reaction, or the probe (e.g., a molecular beacon probe) may be configured to hybridize with the amplicon after the amplification reaction has been completed, among others.

Quenching—any proximity-dependent process that results in a decrease in the photoluminescence of a photoluminophore. Quenching may occur through any suitable mechanism or combination of mechanisms, including dynamic quenching (e.g., Förster Resonance Energy Transfer (FRET), Dexter electron transfer, Exciplex, etc.) and/or static/contact quenching, among others. The efficiency of quenching may be very sensitive to the distance between a photoluminophore and its quencher. For example, in FRET the efficiency of quenching is inversely related to this distance raised to the sixth power. Accordingly, small changes in the separation distance between the photoluminophore and quencher can produce large changes in the efficiency of quenching. The distance at which the quenching efficiency has dropped to 50% may be less than 10 nanometers.

A quencher is a label capable of quenching the photoluminescence of a photoluminophore, generally in a highly proximity-dependent manner. The quencher may be another photoluminophore, or may be a dark quencher that does not substantially emit light. Exemplary dark quenchers may include Black Hole Quenchers (e.g., BHQ0, BHQ1, BHQ2, BHQ3), ATTO quenchers, Iowa Black, QSY 7/9/21/35, etc.

Reference—a target or set of targets that serves as an internal standard to which a target of interest or target set of interest can be compared. Accordingly, a reference typically has a substantially constant (or at least more constant) copy number among the cells or nuclei being tested, such as a copy number of one or two, while a target or target set of interest may have a more variable copy number among the cells or nuclei (e.g., due to duplications and/or deletions).

Set of targets—two or more targets of different sequence that are detected collectively, and optionally indistinguishably. A set of targets, interchangeably referred to as a “target set” (such as a first target set of two or more first targets), may be composed of two or more targets each located in, or expressed from, a copy of the same chromosome, chromosome region, or gene, among others, or at least two of the targets of the set may be located on, or expressed from, different chromosomes. Each target of the set may be amplified, at least initially, with a different pair of primers. Amplification of each target of the set may be reported with a different target-specific probe, with the same probe (e.g., a probe that anneals to the same probe binding site incorporated into each type of amplicon via a primer), with the same intercalating dye, or the like. Amplification of each target of the set of targets may be reported by the same detected signal (also called an amplification-reporting signal). For example, if the signal is detected photoluminescence, the photoluminescence may be detected in the same wavelength regime (e.g., emitted from the same species of photoluminophore present in different probes) for each target of the set of targets.

Target—a nucleic acid sequence (DNA and/or RNA) or protein of any suitable length. Exemplary nucleic acid targets are about 20-1000 nucleotides, or about 30-500 nucleotides, among others. Exemplary protein targets may be detected by a proximity ligation assay (P LA) or a proximity extension assay (PEA). A target interchangeably may be called a target sequence.

Template—a nucleic acid including a sequence that is amplified.

### II. METHOD OVERVIEW

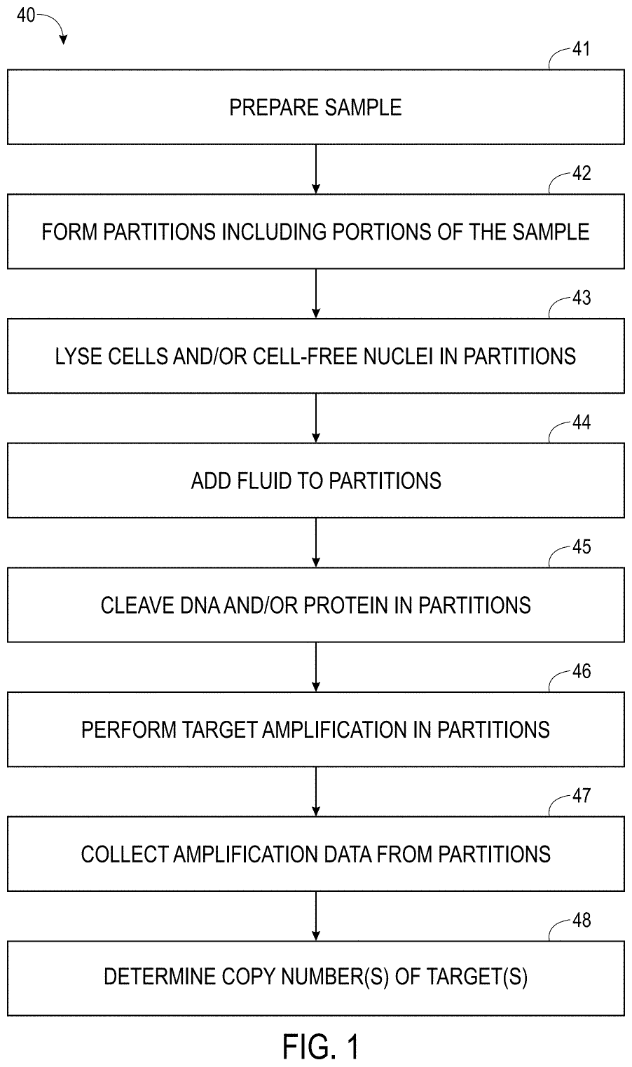

This section provides an overview of partition-based amplification methods of analyzing a sample to determine a copy number of at least one target or set of targets for individual cells or nuclei of the sample; see FIGS. 1-4.

FIG. 1 shows a flowchart 40 of exemplary steps for a partition-based amplification method to determine target copy number for individual cells/nuclei. The steps may be performed in any suitable order and combination for the method, and may be modified by or supplemented with any other disclosure herein.

A sample may be prepared, indicated at 41. The sample includes cells and/or cell-free nuclei of interest, which may be substantially intact. Each cell/nucleus of interest contains at least one copy of at least one target to be assayed in the method. The copy number of one or more targets to be assayed in the method may exhibit copy number heterogeneity among the cells/nuclei of interest. Accordingly, the cells/nuclei of interest may include at least two different types or species of cells/nuclei of interest, such as maternal and fetal, normal and tumor, tumor with target heterogeneity/instability, transgenic with target heterogeneity/instability, or the like. The sample also may contain other cells/nuclei that are not of interest and do not contain the at least one target.

Preparation of the sample may include forming a sample-containing fluid, also called a bulk phase. More specifically, cells/nuclei and other components of the sample (e.g., a surrounding liquid, buffer, salt, debris, etc.) may be combined with one or more lysis reagents, one or more amplification reagents, an aqueous dilution fluid, and/or the like. The amplification reagents may be configured to amplify at least one target or set of targets, and may include a pair of primers for each target, at least one label (e.g., the same label) to report amplification of a target or set of targets, a polymerase/ligase to catalyze target amplification, dNTPs/NTPs, or the like. The aqueous dilution fluid may be added to adjust the number of cells/nuclei per unit volume, to facilitate forming partitions with single cells/nuclei of interest. Further aspects of lysis reagents and amplification reagents that may be suitable are described above in Section I.

Partitions may be formed, indicated at 42. Any suitable number of partitions may be formed and/or utilized, such as at least 10, 25, 50, 100, 200, 500, 1000, 10,000, 100,000, or one million, among others. The partitions may be formed using a sample-containing fluid (or bulk phase) generated in sample preparation step 41, and may be substantially uniform in size. For example, the sample-containing fluid may be divided to form partitions each composed substantially entirely of the sample-containing fluid, and these partitions may be utilized for subsequent steps of the method. In other cases, partitions for use in subsequent steps of the method may be formed by introducing portions of the sample-containing fluid into pre-formed, isolated fluid volumes, such as by pipetting or picoinjection. In yet other cases, the partitions for use in subsequent steps of the method may formed by dividing the sample-containing fluid to create isolated fluid volumes, which are then supplemented with additional fluid before cell/nuclear lysis.

Each partition includes a portion of the sample-containing fluid (and thus a portion of the sample). In some embodiments, each partition of only a subset of the partitions may receive at least one of the cells/nuclei of interest from the sample. In other words, each partition of another subset of the partitions may receive no cell/nucleus of interest from the sample. Optionally, yet another subset of the partitions receives at least two of the cells/nuclei of interest from the sample. Accordingly, the distribution of cells/nuclei to partitions may be substantially stochastic (e.g., generally having a Poisson distribution), if the cells/nuclei are separated from one another (e.g., not aggregated or clumped together) in the sample-containing fluid. In other embodiments, a microfluidic device may be used to increase the percentage of partitions that have exactly one cell or nucleus. For example, if the partitions are droplets, the microfluidic device may trigger droplet formation when and only when a cell or nucleus is present, thus permitting essentially every droplet to contain only one cell or nucleus.

In some embodiments, fewer than one-half of the partitions may contain at least one cell (or cell-free nucleus). For example, fewer than about 30%, 20%, or 10% of the partitions may contain at least one cell or cell-free nucleus. If individual cells/nuclei are assumed to localize to partitions independently of one another when the partitions are formed, the frequency at which two or more cells (and/or cell-free nuclei) colocalize to the same partition by chance can be kept to a substantially negligible level if the percentage of cell/nucleus-free partitions is relatively high. For example, if only about 10% of the partitions receive at least one cell/nucleus, then statistically only about 1% of the partitions would be expected to receive two cells/nuclei by chance colocalization.

In other embodiments, the frequency of partitions containing two or more cells may be significant. For these embodiments, partitions with more than one cell/nucleus may be identified in the method (and optionally eliminated from any contribution to the final result of the analysis), as described below.

Cells/nuclei in the partitions may be lysed, indicated at 43. Lysis may be encouraged by any suitable combination of physical and/or chemical treatments. For example, the partitions may be heated above room temperature, such as to a temperature of at least 37, 40, 50, 60, 70, 80, 85, 90, or 95 degrees Celsius. Heating may be conducted for any suitable length of time, such as 1-120, 2-90, or 3-60 minutes, or for at least about 1, 2, 3, 5, 10, 20, 30, 45, or 60 minutes. Each partition may include a nonionic or ionic surfactant to encourage lysis and/or improve accessibility to target sequences. Further aspects of cell/nuclear lysis are described above in Section I and elsewhere in the present disclosure.

Fluid optionally may be added to the partitions, indicated at 44. The fluid may be liquid and may contain any suitable reagents, and the same volume of the fluid may be added to each partition. The fluid may carry a reagent, such as a heat-sensitive (and/or lysis-sensitive) reagent (e.g., a nuclease, protease, polymerase, and/or ligase), or may dilute enzyme-inhibiting substances present in the partitions to reduce their inhibitory activity. The fluid may be added by picoinjection using an electric field, by pipetting, or the like. Fluid addition step 44 may increase the size of each partition substantially (e.g., a volume increase of at least about 50%, 100%, or 200%, among others), or may increase the volume of each partition by less than about 50%.

DNA and/or protein in the partitions may be cleaved, indicated at 45. This cleavage may be limited and selective. Accessibility to target sequences may be promoted by cleavage of nucleic acid with a nuclease and/or cleavage of protein with a protease, to ensure that each copy of each target is accessible to amplification reagents when amplification begins. The cleavage may be catalyzed by incubation at a relatively lower temperature at which the enzyme(s) is active (e.g., 37 degrees Celsius) after incubation at a relatively higher lysis temperature (e.g., 80 degrees Celsius). One or more foreign (exogenous) cleavage enzymes to catalyze the cleavage may be added in fluid-addition step 44, or may be present when the partitions are formed (e.g., included in the sample-containing fluid).

Target amplification may be performed in the partitions, indicated at 46. Any suitable number of different targets and/or different sets of targets may be amplified, to produce amplicons corresponding to the targets and/or sets of targets. A different amplification reaction may be performed for each target. Target amplification may be promoted by heating the partitions to a fixed incubation temperature for isothermal amplification, or thermally cycling the partitions between/among different temperatures, for amplification by PCR (polymerase chain reaction) or LCR (ligase chain reaction), among others. The different temperatures may include a denaturation temperature, an annealing temperature, and an extension temperature; a denaturation temperature and an annealing/extension temperature; or the like.

The amplification reactions may be stopped before the amplification endpoint of any of the reactions is reached. More specifically, each amplification reaction may be stopped in an exponential/linear phase of amplification. However, stopping in the exponential phase is generally preferred, because in this phase the number of copies of each type of amplicon more accurately and sensitively reflects the initial copy number of each corresponding target in individual partitions (and in single cells/nuclei lysed in these partitions).

Amplification data may be collected from the partitions, indicated at 47. The amplification data may be collected before any of the amplification reactions have reached an endpoint, such as when each amplification reaction is in an exponential/linear phase of amplification. In some embodiments, all of the amplification data may be collected after completion of the same number of thermal cycles, optionally, a predefined number of thermal cycles. In other embodiments, the amplification data may be collected from the partitions at multiple time points, such as after completion of each of two or more different numbers of thermal cycles (e.g., if the optimum number of cycles for distinguishing different copy numbers of target is not known). In yet other embodiments, for isothermal amplification, all of the amplification data may be collected after the same duration of isothermal incubation or at two or more different time points after the start of isothermal incubation.

Amplification data may be collected by detecting one or more signals (amplification-reporting signals) from each of the partitions. The one or more signals may be detected from at least one label present in each of the partitions. In some embodiments, each signal is detected from a different species of label and represents a different target or set of targets. Since the amplification data is collected before the amplification endpoint is reached, the amplitude (magnitude) of each signal varies, directly or inversely, according to the initial copy number of each corresponding target or set of corresponding targets in individual partitions. The degree to which the amplitude varies is generally greatest during the exponential phase of amplification, once sufficient amplification has occurred to distinguish target-positive partitions from target-negative partitions.

Each amplification-reporting signal may represent photoluminescence, such as fluorescence, detected from the partitions. The intensity of the photoluminescence for each partition may correspond to the initial copy number of a target or set of targets in the partition (and thus in at least one cell/nucleus, if any, initially present in the partition when formed). A distinguishable photoluminescence may be detected to measure amplification of each different target or set of targets. For example, the photoluminescence may be detected in different wavelength regimes for different targets/sets of targets from corresponding different labels. For example, a first species of photoluminophore may label a first probe or first set of probes to produce a first photoluminescence, and a second species of photoluminophore may label a second probe or second set of probes to produce a second photoluminescence, where the first photoluminescence and the second photoluminescence represent different wavelengths from one another (e.g., different colors of emitted light).

One or more copy numbers of each target or set of targets may be determined using the amplification data. For example, partitions may be assigned to different groups (also called clusters) having similar (clustered) values for at least one amplification-reporting signal. Each group may be assigned a different copy number for the target or set of targets, where partitions within the group are assigned the same copy number. The copy number may be a whole number, such as 0, 1, 2, 3, etc. In some cases, each partition may be excluded for which the at least one amplification-reporting signal indicates the partition received none or more than one of the cells/nuclei from the sample. In some cases, the sample may be a test sample, and determining a copy number includes comparing values for the at least one amplification-reporting signal to corresponding values obtained with a control sample including cells or cell-free nuclei having a known copy number of the target or set of targets.

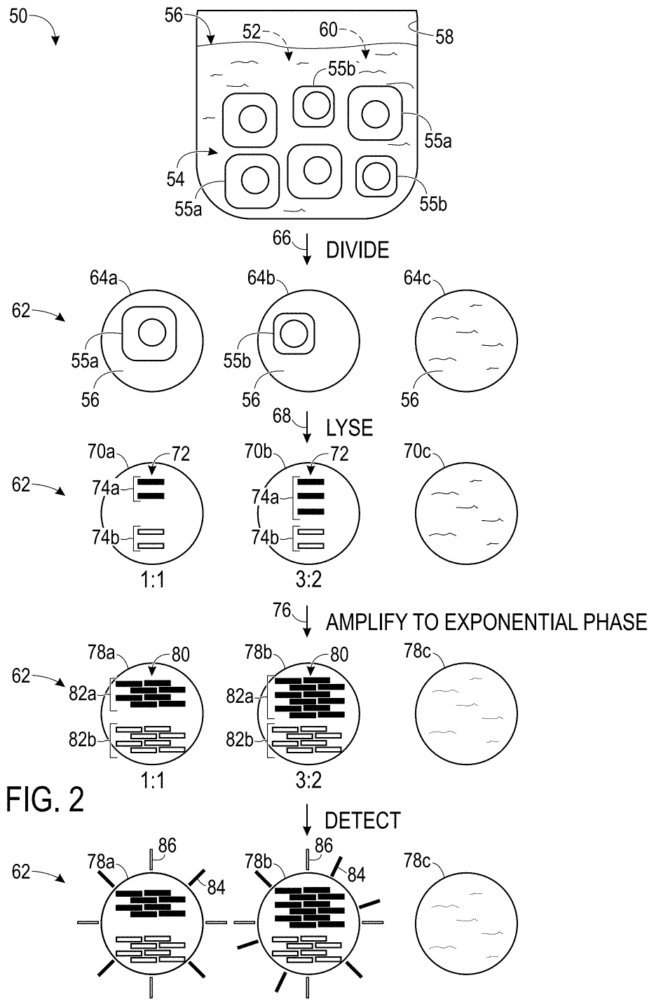

FIG. 2 schematically illustrates aspects of an exemplary partition-based amplification method 50 of analyzing a sample 52 including cells 54 and/or cell-free nuclei. Method 50 may include any suitable combination of steps 41-48 (see FIG. 1), but only a subset of these steps are illustrated in FIG. 2. The method is being utilized here for NIPT (noninvasive prenatal testing), where sample 52 is obtained from a pregnant female, and cells 54 include a mixture of maternal cells 55a and fetal cells 55b. Fetal cells 55b in sample 52 are trisomic for one of the two chromosomes being assayed by this embodiment of the method, while maternal cells 55a are disomic for both of the two chromosomes.

A sample-containing fluid 56 may be prepared, such as in a vessel 58. Sample-containing fluid 56 may be aqueous liquid including sample 52, which may contain cells 54 and/or cell-free nuclei. Sample-containing fluid 56 also may contain lysis/amplification reagents 60.

Partitions 62 may be formed, indicated by an arrow at 66. For example, the bulk phase of sample-containing fluid 56 may be divided to form partitions 62 of substantially the same volume. Only three illustrative pre-lysis partitions 64a-c are shown here, to simplify the presentation, and are kept in the same order for each subsequent step of method 50, to distinguish the effect of the step on each different partition. However, any suitable number of partitions 62 may be formed to obtain a desired level of statistical confidence in the results of the method.

Partitions 62 may contain different numbers of cells/nuclei. A plurality of partitions 62, represented by pre-lysis partition 64c, each may contain no cells 54 (or no cell-free nuclei). Another plurality of partitions 62, represented by pre-lysis partitions 64a and 64b, each may contain a single cell 54 (or a single cell-free nucleus) from sample 52. In some embodiments, yet another plurality of partitions 62 each may contain at least two cells/nuclei (not shown).

Cells 54 and/or cell-free nuclei in partitions 62 may be lysed, indicated at 68, to produce post-lysis partitions 70a-c from pre-lysis partitions 64a-64c, respectively. Lysis may release and/or expose one or more copies of at least one target (or set of targets (i.e., a target set)) 72 to be detected and quantified for individual partitions 62. Here, a pair of different targets (or target sets) 74a, 74b are shown as released by lysis of maternal cell 55a in post-lysis partition 70a and lysis of fetal cell 55b in post-lysis partition 70b. Each target 74a, 74b represents a different chromosome in cells 54. Target 74a represents a chromosome that is disomic in maternal cells 55a and trisomic in fetal cells 55b. Target 74b represents a chromosome that is disomic in both types of cells 55a, 55b.

No copies of first target 74a (or second target 74b) are present in post-lysis partition 70c, which did not receive either type of cell (55a or 55b) from sample-containing fluid 56. Two and three copies of first target 74a are present in post-lysis partitions 70a and 70b, respectively (i.e., two copies from maternal cell 55a and three copies from fetal cell 55b). In other words, the copy number of first target 74a is two, three, and zero in post-lysis partitions 70a, 70b, and 70c, respectively, and thus varies among cells 54 between at least two values (i.e., two and three). Each of post-lysis partitions 70a and 70b contains two copies of second target 74b. Accordingly, in the two cells being tested, first target 74a exhibits copy number variation, while second target 74b does not. The ratios of copy numbers for the first and second targets 74a, 74b are given under post-lysis partitions 70a and 70b, as 1:1 and 3:2 respectively.

One or more amplification reactions may be performed in post-lysis partitions 70a-c, indicated at 76, to produce amplified partitions 78a-c, respectively. The amplification reaction(s) generates amplicon(s) 80 corresponding to one or more targets 72 being amplified. A different amplification reaction may be performed for each target 72 to generate a corresponding amplicon. For example, here, amplification of first target 74a and second target 74b generates copies of two types of amplicons, 82a and 82b, respectively. In other cases, a set of targets may be amplified for each copy number to be determined. For example, amplicon 82a (and/or 82b) may be a set of different amplicons corresponding to a set of targets 74a (or 74b).

The amplification reactions may be stopped before the amplification endpoint is reached. More specifically, each amplification reaction may be stopped in an exponential/linear phase of amplification. However, stopping in the exponential phase is generally preferred, because in this phase the number of copies of each type of amplicon 82a, 82b more accurately and sensitively reflects the initial copy number of each corresponding target in individual partitions (and single cells/nuclei). For example, the ratios of amplicon 82a to amplicon 82b in amplified partitions 78a and 78b may be about the same as the ratio of first target 74a to second target 74b in cells 54 of pre-lysis partitions 64a and 64b.

Amplification data may be collected by detecting one or more signals from the partitions. Here, distinguishable first and second photoluminescence 84, 86 is detected at different wavelengths from the partitions, optionally from only one species or two or more different species of photoluminophore for each target (or target set) 74a, 74b. The intensity of first and second photoluminescence 84, 86 detected from amplified partitions 78a, 78b corresponds to the initial copy number of first and second targets 74a, 74b in post-lysis partitions 70a, 70b. The intensity of first photoluminescence 84 detected from amplified partition 78b is significantly higher than from amplified partition 78a, because the copy number of first target 74a is 50% higher in post-lysis partition 70b than post-lysis partition 70a. In contrast, the intensity of second photoluminescence 86 detected from amplified partitions 78a, 78b is substantially the same, because the copy number of second target 74b is the same in post-lysis partitions 70a, 70b.

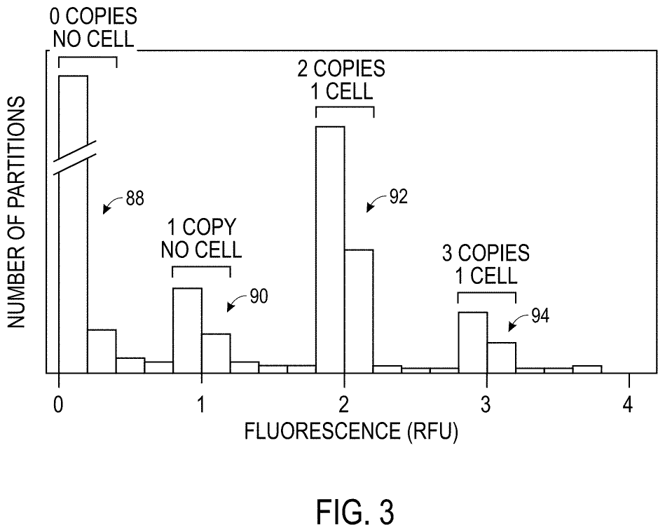

FIG. 3 shows a conceptual histogram illustrating exemplary amplification data that may be collected from partitions 62 for amplification of target 74a in method 50 (also see FIG. 2). The amplification data may be detected as fluorescence intensity (i.e., first photoluminescence 84) from each partition 62. The histogram has a fluorescence axis divided into intensity intervals. The number of partitions 62 having a fluorescence intensity value falling within each intensity interval is represented with a bar having a height proportional to the number.

Four different groups 88, 90, 92 and 94 of partitions having distinct fluorescence intensities are identifiable in the histogram. Each group represents a different number of copies of first target 74a present initially in individual partitions 62. No-copy group 88 received no cell and no copy of target 74a. Group 88 may contain substantially more partitions than the other groups to reduce the incidence of multiple cells colocalizing to the same partition. One-copy group 90 received no cell and only one copy of a cell-free (and nucleus-free) form of first target 74a. The frequency of partitions in one-copy group 90 may be related to the quality of sample 52 and the amount of premature cell lysis, if any, that occurs before partitions 62 are formed. Two-copy group 92 received one maternal cell 55a, which contained two copies of first target 74a (i.e., the maternal cell is disomic for the chromosome providing first target 74a). Three-copy group 94 received only one fetal cell 55b, which contained three copies of first target 74a (i.e., the fetal cell is trisomic for the chromosome providing first target 74a).

In some cases, partitions containing one disomic cell (two copies of first target 74a) may also receive a third copy of the first target from a prematurely lysed cell. These partitions introduce error into the assay because they falsely appear to represent trisomic cells. However, the frequency of these potential false-positive trisomic partitions can be minimized by using a first target set (from a single chromosome or from two, three, or more different chromosomes) rather than a single first target. The use of a first target set may increase the level of noise, because a higher percentage of partitions receive one or more targets, but may reduce the number of erroneous copy-number assignments.

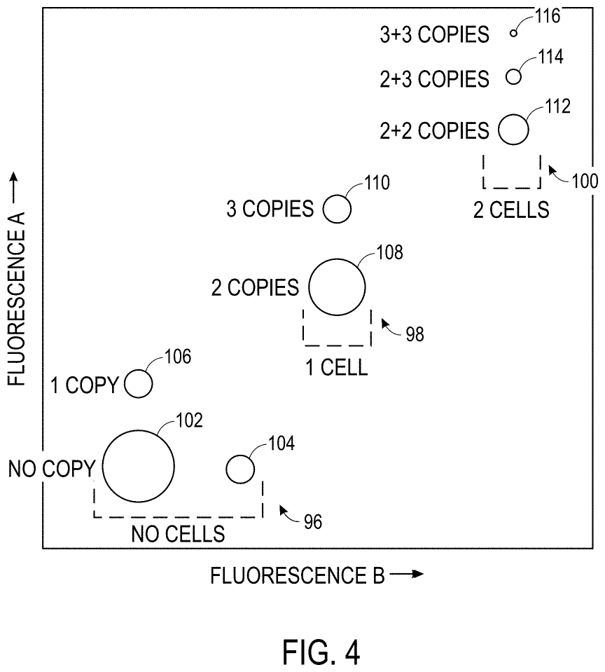

FIG. 4 shows a conceptual two-dimensional scatterplot illustrating exemplary amplification data that may be collected from partitions 62 for amplification of first and second targets 74a and 74b in method 50 (also see FIG. 2). The amplification data may be detected as fluorescence intensity in different wavelength regimes from each partition 62. First photoluminescence 84 (fluorescence A) corresponds to first target 74a, and second photoluminescence 86 (fluorescence B) corresponds to second target 74b. Each partition is represented by a point in the scatterplot, but individual points are not shown here. Instead, each identifiable cluster of points is represented as a group by a circle around the cluster.

The data of FIG. 4 may result when method 50 is performed with a higher ratio of cells 54 to partitions 62, such that a significant percentage of the partitions receive two cells 54. Three sets of clusters having distinct fluorescence intensities are identifiable in the scatterplot, each representing partitions that contained a different number of cells 54 when formed: no-cell set 96, one-cell set 98, and two-cell set 100. Each cluster of partitions within a set can be described as a group. The number of copies of first target 74a in each group is listed.

No-cell set 96 is composed of groups 102, 104, and 106. Partitions of double-negative group 102 contained no copy of first target 74a and no copy of second target 74b. Partitions of single copy group 104 initially contained no copy of first target 74a and one copy of (cell-free) second target 74b. Partitions of single-copy group 106 initially contained no copy of second target 74b and one copy of (cell-free) first target 74a.

One-cell set 98 is composed of groups 108 and 110. Partitions of maternal group 108 initially contained two copies of first target 74a (and two copies of second target 74b) provided by a maternal cell 55a. Partitions of fetal group 110 initially contained three copies of first target 74a (and two copies of second target 74b) provided by a fetal cell 55b.

Two-cell set 100 is composed of groups 112, 114, and 116. Partitions of double maternal group 112 initially contained two maternal cells 55a, each providing two copies of first target 74a (i.e., 2+2 copies). Partitions of maternal-fetal group 114 initially contained one maternal cell 55a and one fetal cell 55b, respectively providing two copies and three copies of first target 74a (i.e., 2+3 copies). Partitions of double fetal group 116 initially contained two fetal cells 55b, each providing three copies of first target 74a (i.e., 3+3 copies).

### III. EXAMPLES

This section describes additional aspects of the present disclosure related to partition-based determination of target copy number for single cells by non-endpoint amplification. These aspects are intended for illustration and should not limit the entire scope of the present disclosure.

### Example 1. Fluorescence of Droplets Containing a Series of Dye Concentrations

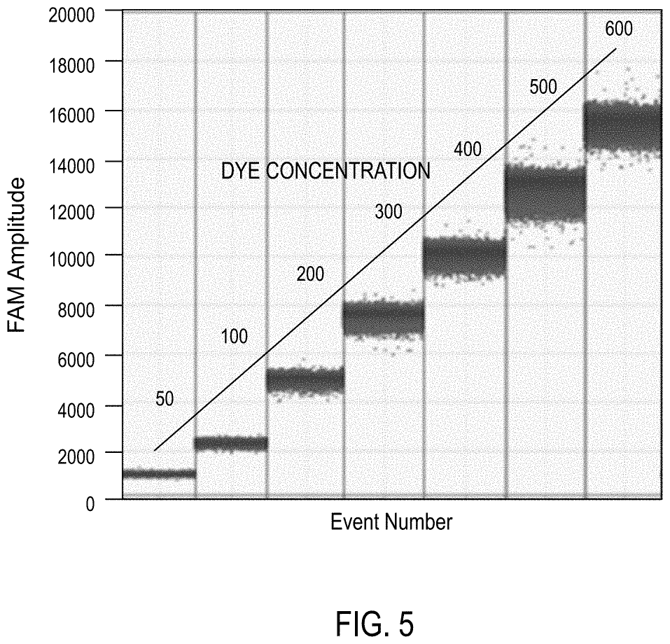

FIG. 5 shows a graph plotting the intensity of FAM fluorescence measured from seven separate sets of droplets each pre-loaded with a different amount of FAM dye (i.e., to achieve a dye concentration of 50 nM, 100 nM, 200 nM, etc., as indicated). The droplets exhibit a fluorescence intensity that is substantially proportional to the dye concentration. Different dye concentrations are clearly distinguishable from one another over a 12-fold range, and even a 20% difference in dye concentration (500 versus 600) is resolved.

### Example 2. Amplification Cycle Dependence of Fluorescence Intensity from Droplets

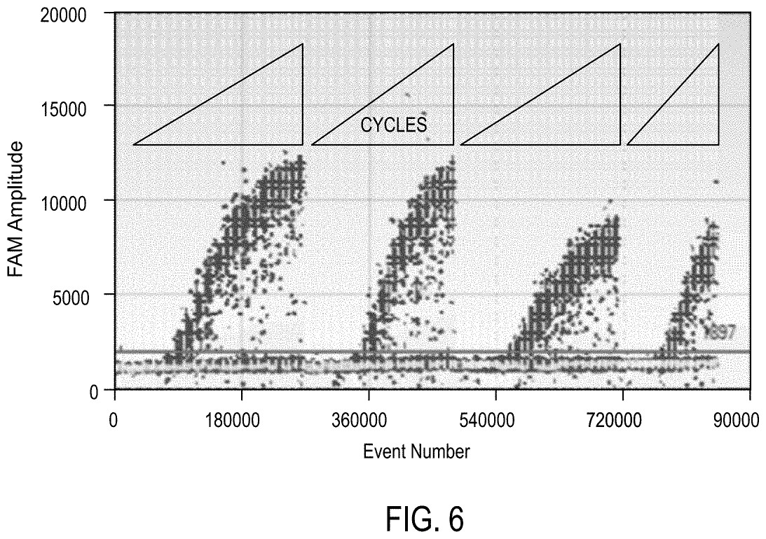

FIG. 6 shows a graph plotting the amplitude of FAM fluorescence, as an indicator of target amplification by PCR, detected from individual droplets (events) as the number of PCR cycles increases, for four separate sets of droplets.

### Example 3. Effect of Template Topology on Target Amplification

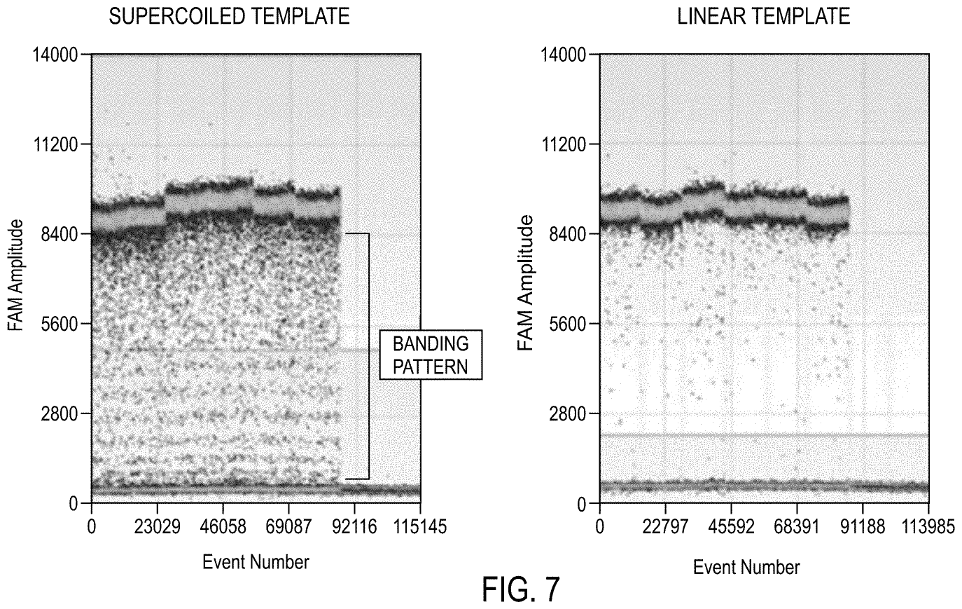

FIG. 7 shows a pair of graphs comparing droplet-based PCR amplification assays using a supercoiled template (on the left) or a linearized template (on the right) as the source of the same target sequence. The amplitude of FAM fluorescence is detected from a series of droplets (events), and the heavy band of highest fluorescence represents droplets that have reached the amplification endpoint for the target sequence.

A banding pattern of lighter bands is visible with the supercoiled template but not the linearized template. This banding pattern may be produced because target amplification from the supercoiled template is inefficient until the template is nicked during thermal cycling. Each successive band of increasing FAM amplitude may represent a successively earlier cycle in which the supercoiled template was nicked, but not early enough for target amplification to reach an endpoint. These data indicate that providing efficient access to the target sequence(s) before the start of the amplification reaction may produce more tightly clustered amplification signals for each type of partition, and thus more accurate assignment of partition types and determination of copy numbers.

### Example 4. Allelic Combinations of N-RAS Detectable in Droplets

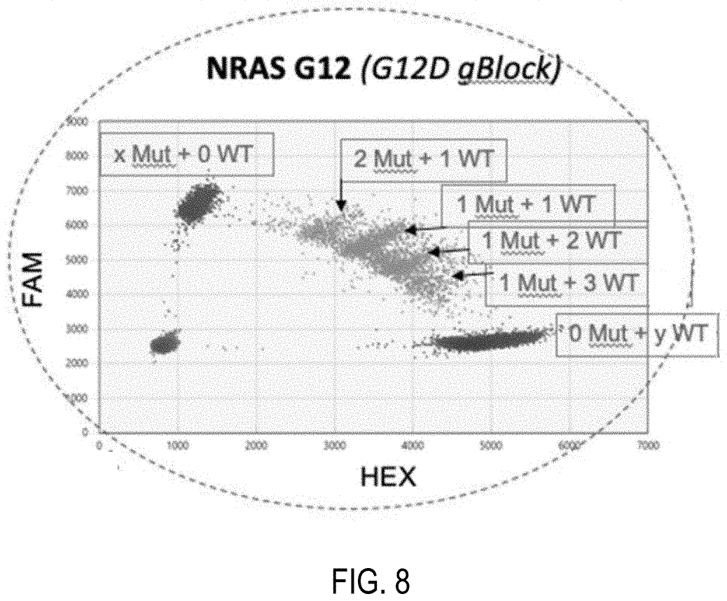

FIG. 8 shows a two-dimensional fluorescence scatterplot of amplification data collected from droplets containing wild-type (WT) and mutant (G12D) N-RAS target sequences in various combinations. The wild-type and mutant target sequences are detected as increases in HEX and FAM fluorescence, respectively. These data are significant because they reflect the ability of the droplet-based amplification system to distinguish droplet clusters containing different ratios of two distinguishable targets by differences in their 2D fluorescence amplitudes.

### IV. SELECTED ASPECTS

This section describes selected aspects of the present disclosure as a series of indexed paragraphs.

A1. A method of analyzing a sample including cells and/or cell-free nuclei, the method comprising: (a) forming partitions each including a portion of the sample, wherein each partition of at least a subset of the partitions contains only one of the cells/nuclei from the sample; (b) lysing cells and/or cell-free nuclei from the sample in the partitions; (c) performing at least one amplification reaction for a target or set of targets in the partitions; (d) collecting amplification data from the partitions in an exponential/linear phase of each amplification reaction; and (e) determining a copy number of the target or set of targets for individual partitions using the amplification data.

A2. The method of paragraph A1, wherein performing at least one amplification reaction includes thermally cycling the partitions for a predefined number of cycles, and wherein all of the amplification data used for determining a copy number of the target or set of targets represent completion of the same predefined number of cycles.

A3. The method of paragraph A1 or A2, wherein the cells/nuclei include a first population of one or more cells/nuclei having a first copy number for the target or set of targets and a second population of one or more cells/nuclei having a second copy number for the target or set of targets, the method further comprising enumerating partitions of the first population and partitions of the second population.

A4. The method of paragraph A3, wherein the first population has a copy number of two for the target or set of targets, and wherein the second population has a copy number of one, or has a copy number of at least three for the target or set of targets.

A5. The method of paragraph A4, wherein the second population has a copy number of three for the target or set of targets.

A6. The method of any of paragraphs A1 to A5, wherein collecting amplification data includes detecting photoluminescence from the partitions, and wherein an intensity of the photoluminescence varies among the partitions according to the copy number of the target or set of targets in individual partitions.

A7. The method of paragraph A6, wherein detecting photoluminescence includes detecting fluorescence.

A8. The method of any of paragraphs A1 to A7, wherein the target or set of targets is a single target.

A9. The method of any of paragraphs A1 to A7, wherein the target or set of targets is a set of two or more targets.

A10. The method of paragraph A9, wherein collecting amplification data includes detecting photoluminescence having an intensity that varies among the partitions according to the copy number of the set of targets in individual partitions.

A11. The method of paragraph A9 or A10, wherein each target of the set of targets represents the same chromosome in the cells/nuclei.

A12. The method of any of paragraphs A1 to A11, wherein the target or set of targets represents human chromosome 13, 18, 21, X, or Y in the cells/nuclei.

A13. The method of any of paragraphs A1 to A12, wherein the target or set of targets is a first target or first set of targets, wherein performing at least one amplification reaction includes performing at least one amplification reaction for a second target or second set of targets, and wherein determining includes determining a copy number of the second target or second set of targets for individual partitions.

A14. The method of paragraph A13, wherein the first target or first set of targets represents a first chromosome in the cells/nuclei, and wherein the second target or second set of targets represents a different, second chromosome in the cells/nuclei, and wherein, optionally, the second chromosome is a reference chromosome that is statistically less susceptible (e.g., not normally susceptible) to aneuploidy than the first chromosome during fetal development.

A15. The method of paragraph A14, wherein the first chromosome is selected from human chromosomes 13, 18, 21, X, and Y.

A16. The method of paragraph A14 or A15, wherein the second chromosome is human chromosome 1.

A17. The method of any of paragraphs A13 to A16, wherein collecting amplification data includes detecting a first photoluminescence having an intensity corresponding to amplification of the first target or first set of targets and a second photoluminescence having an intensity corresponding to amplification of the second target or second set of targets

A18. The method of any of paragraphs A1 to A17, wherein the cells/nuclei of the sample include maternal cells/nuclei and fetal cells/nuclei.

A19. The method of any of paragraphs A1 to A18, further comprising enumerating cells/nuclei having an abnormal copy number of the target or set of targets.

A20. The method of paragraph A19, further comprising enumerating cells/nuclei having a normal copy number of the target or set of targets.

A21. The method of any of paragraphs A1 to A20, further comprising identifying partitions that contained no intact cell or nucleus when formed, based on the amplification data.

A22. The method of paragraph A21, wherein collecting amplification data includes detecting a signal from each partition, wherein identifying partitions includes comparing the signal from individual partitions with a threshold, and wherein individual partitions for which the signal is less than the threshold are identified as having contained no cell or nucleus from the sample when formed.

A23. The method of any of paragraphs A1 to A22, wherein the cells/nuclei of the sample include tumor cells/nuclei.

A24. The method of any of paragraphs A1 to A23, wherein the cells/nuclei of the sample include transgenic cells/nuclei.

A25. The method of paragraph A24, wherein the transgenic cells/nuclei contain two or more different copy numbers of an inserted nucleotide sequence including the target or set of targets.

A26. The method of paragraph A25, wherein the transgenic cells/nuclei are from a first sample obtained at a first time point from a transgenic source, and wherein forming, lysing, performing, collecting, and determining are conducted again at least once using at least a second sample obtained at a later, second time point from the transgenic source, to measure instability, if any, of the inserted nucleotide sequence.

A27. The method of any of paragraphs A1 to A26, wherein the target or set of targets includes an RNA target sequence or a DNA target sequence.

A28. The method of any of paragraphs A1 to A27, further comprising exposing nucleic acid of the cells/nuclei to an exogenous nuclease during and/or after lysing.

A29. The method of any of paragraphs A1 to A28, further comprising exposing proteins of the cells/nuclei to an exogenous protease during and/or after lysing.

A30. The method of any of paragraphs A1 to A29, wherein lysing includes heating the partitions to at least 50, 60, 70, 80, 85, or 90 degrees Celsius.

A31. The method of paragraph A30, wherein heating includes heating the partitions for about 1-120, 2-90, or 3-60 minutes.

A32. The method of paragraph A30 or A31, wherein heating includes heating the partitions for at least about 1, 2, 3, 5, 10, 20, 30, 45, or 60 minutes.

A33. The method of any of paragraphs A1 to A32, wherein lysing includes exposing the cells/nuclei to a surfactant.

A34. The method of any of paragraphs A1 to A33, wherein forming partitions includes dividing the same sample-containing fluid into aqueous droplets surrounded by an immiscible liquid.

A35. The method of paragraph A34, wherein the immiscible liquid includes oil.

A36. The method of any of paragraphs A1 to A35, wherein each partition includes a portion of the same sample-containing first fluid, the method optionally further comprising adding a second fluid to the partitions after lysing.

A37. The method of paragraph A36, wherein adding a second fluid includes picoinjecting the second fluid into partitions, optionally using an electric field.

A38. The method of paragraph A36, wherein adding a second fluid includes pipetting the second fluid into separate compartments each holding only one of the partitions, and wherein the separate compartments include wells, nanochambers, or microtubes.

A39. The method of any of paragraphs A1 to A38, wherein performing at least one amplification reaction includes performing PCR.

A40. The method of any of paragraphs A1 to A39, wherein the partitions when formed contain an average of less than one cell/nucleus from the sample per partition.

A41. The method of any of paragraphs A1 to A40, wherein a plurality of the partitions do not contain at least one of the cells/nuclei.

A42. The method of any of paragraphs A1 to A41, wherein collecting amplification data includes detecting at least one amplification-reporting signal from the partitions, and wherein determining a copy number includes identifying a group of the partitions having clustered values for the at least one amplification-reporting signal and assigning the same copy number to each partition of the group.

A43. The method of any of paragraphs A1 to A42, wherein the sample is a test sample, wherein collecting amplification data includes detecting at least one amplification-reporting signal from the partitions, and wherein determining a copy number includes comparing values for the at least one amplification-reporting signal to corresponding values obtained with a control sample including cells or cell-free nuclei having a known copy number of the target or set of targets.

A44. The method of paragraph A43, wherein the known copy number is a whole number.

A45. The method of any of paragraphs A42 to A44, wherein determining a copy number includes identifying a first group and a second group of the partitions based on the at least one amplification-reporting signal, wherein the first group and the second group are assigned respective first and second copy numbers for the target or set of targets, and wherein the first and second copy numbers are different from one another.

A46. The method of paragraph A45, wherein the first and second copy numbers are whole numbers.

A47. The method of any of paragraphs A1 to A46, wherein collecting amplification data includes detecting two or more distinct amplification-reporting signals from the partitions, wherein each of the two or more distinct amplification-reporting signals represents a different target or set of targets in the cells/nuclei.

A48. The method of paragraph A47, wherein each of the two or more amplification-reporting signals represents a different chromosome in the cells/nuclei.

A49. The method of any of paragraphs A1 to A48, wherein collecting amplification data includes detecting at least one amplification-reporting signal from the partitions, and wherein determining a copy number includes excluding each partition for which the at least one amplification-reporting signal indicates the partition received none or more than one of the cells/nuclei from the sample.

A50. The method of any of paragraphs A42 to A49, wherein each amplification-reporting signal is detected as photoluminescence from the partitions.

A51. The method of any of paragraphs A1, A3-A38, and A40-A50, wherein performing at least one amplification reaction includes performing at least one isothermal amplification reaction.

A52. The method of any of paragraphs A1 to A51, further comprising comparing the copy number to at least one threshold, and diagnosing aneuploidy or cancer if comparing meets one or more predefined criteria.

A53. The method of any of paragraphs A1 to A52, further comprising comparing the copy number to at least one threshold, and administering a treatment if comparing meets one or more predefined criteria.

The term “exemplary” as used in the present disclosure, means “illustrative” or “serving as an example.” Similarly, the term “exemplify” means “to illustrate by giving an example.” Neither term implies desirability or superiority.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure. Further, ordinal indicators, such as first, second, or third, for identified elements are used to distinguish between the elements, and do not indicate a particular position or order of such elements, unless otherwise specifically stated.

We claim:

1. A method of analyzing a sample including cells and/or cell-free nuclei, the method comprising:
forming partitions each including a portion of the sample, wherein each partition of at least a subset of the partitions contains only one of the cells/nuclei from the sample;
lysing cells and/or cell-free nuclei from the sample in the partitions;
performing at least one amplification reaction for a target or set of targets in the partitions;
collecting amplification data from the partitions in an exponential/linear phase of each amplification reaction; and
determining a copy number of the target or set of targets for individual partitions using the amplification data.

2. The method of claim 1, wherein performing at least one amplification reaction includes thermally cycling the partitions for a predefined number of cycles, and wherein all of the amplification data used for determining a copy number of the target or set of targets represent completion of the same predefined number of cycles.

3. The method of claim 1, wherein the cells/nuclei include a first population of one or more cells/nuclei having a first copy number for the target or set of targets and a second population of one or more cells/nuclei having a second copy number for the target or set of targets, the method further comprising enumerating partitions of the first population and partitions of the second population.

4. The method of claim 3, wherein the first population has a copy number of two for the target or set of targets, and wherein the second population has a copy number of one, or has a copy number of at least three for the target or set of targets.

5. The method of claim 4, wherein the second population has a copy number of three for the target or set of targets.

6. The method of claim 1, wherein collecting amplification data includes detecting photoluminescence from the partitions, and wherein an intensity of the photoluminescence varies among the partitions according to the copy number of the target or set of targets in individual partitions.

7. The method of claim 1, wherein the target or set of targets is a single target.

8. The method of claim 1, wherein the target or set of targets is a set of two or more targets.

9. The method of claim 8, wherein collecting amplification data includes detecting photoluminescence having an intensity that varies among the partitions according to the copy number of the set of targets in individual partitions.

10. The method of claim 8, wherein each target of the set of targets represents the same chromosome in the cells/nuclei.

11. The method of claim 1, wherein the target or set of targets is a first target or first set of targets, wherein performing at least one amplification reaction includes performing at least one amplification reaction for a second target or second set of targets, and wherein determining includes determining a copy number of the second target or second set of targets for individual partitions.

12. The method of claim 11, wherein the first target or first set of targets represents a first chromosome in the cells/nuclei, and wherein the second target or second set of targets represents a different, second chromosome in the cells/nuclei.

13. The method of claim 12, wherein the second chromosome is a reference chromosome that is statistically less susceptible to aneuploidy than the first chromosome during fetal development.

14. The method of claim 1, wherein the cells/nuclei of the sample include maternal cells/nuclei and fetal cells/nuclei.

15. The method of claim 1, further comprising enumerating cells/nuclei having an abnormal copy number of the target or set of targets.

16. The method of claim 15, further comprising enumerating cells/nuclei having a normal copy number of the target or set of targets.

17. The method of claim 1, further comprising identifying partitions that contained no intact cell or nucleus when formed, based on the amplification data.

18. The method of claim 1, wherein forming partitions includes dividing the same sample-containing fluid into aqueous droplets surrounded by an immiscible liquid.

19. The method of claim 1, wherein each partition includes a portion of the same sample-containing first fluid, the method further comprising adding a second fluid to the partitions after lysing.

20. The method of claim 1, wherein the partitions when formed contain an average of less than one cell/nucleus from the sample per partition.

21. The method of claim 1, wherein collecting amplification data includes detecting at least one amplification-reporting signal from the partitions, and wherein determining a copy number includes identifying a group of the partitions having clustered values for the at least one amplification-reporting signal and assigning the same copy number to each partition of the group.

22. The method of claim 1, further comprising comparing the copy number to at least one threshold, and diagnosing aneuploidy or cancer if comparing meets one or more predefined criteria.
